(19) United States
(12) Patent Application Publication
(10) Pub. No.: US 2026/0014006 A1
(43) Pub. Date: Jan. 15, 2026

(54) EMBOLIC COIL IMPLANT SYSTEM

(71) Applicant: DeepIn Technologies, LLC, San Jose, CA (US)

(72) Inventors: Ross Soltanian, San Jose, CA (US); Phu Phung, Union City, CA (US)

(73) Assignee: DeepIn Technologies, LLC, San Jose, CA (US)

(21) Appl. No.: 19/332,270

(22) Filed: Sep. 18, 2025

Related U.S. Application Data
(62) Division of application No. 18/072,767, filed on Dec. 1, 2022.
(60) Provisional application No. 63/295,337, filed on Dec. 30, 2021.

(51) Int. Cl. A61F 2/88

(52) U.S. Cl. CPC A61F 2/88

(57) ABSTRACT

An embolic device includes a microcoil having a portion comprising one or more sets of loops. Loops of each of the one or more sets intersect one with another in succession in a cycle, collectively forming a three-dimensional shape. A shape-setting device includes a three-dimensional (3D) body having a curved surface and a plurality of winding members extending from the curved surface of the 3D body and arranged in a plurality of groups. Winding members of each of the plurality of groups are configured to allow a microcoil to wrap around to form a loop, and adjacent groups of winding members share a winding member to allow loops formed by the adjacent groups to intersect one with another. A method of making an embolic device is also provided.

### CROSS-REFERENCE TO RELATED APPLICATIONS

This application is a division of U.S. application Ser. No. 18/072,767 filed Dec. 1, 2022, entitled “Embolic Coil Implant System,” which claims priority to U.S. provisional patent application No. 63/295,337 filed Dec. 30, 2021 entitled “Embolic Coil Implant System,” the disclosures of all of which are hereby incorporated by reference in its entirety.

### TECHNICAL FIELD

This application relates generally to medical devices and methods of making and using medical devices. In particular, various embodiments of an embolic device or coil system for deployment within the vasculature of a human body and a method of making and/or using the coil system are described.

### BACKGROUND

Implants such as embolic devices are known in treatment of vascular disorders such as aneurysms. An aneurysm is a bulge or swelling formed on a wall of an artery in the brain or other locations of a human body. A brain aneurysm can cause severe pain, and if ruptured, lead to fetal stroke. In a non-invasive or minimally invasive treatment of aneurysms, an embolic device may be placed in or at the aneurysm to isolate the aneurysm from blood flow, and/or, promote thrombus formation at the site. The placement of an embolic device is typically accomplished using a delivery system, which steers the embolic device through the vasculature of the patient to the location of the aneurysm. Once positioned at or in the aneurysm, the embolic device is detached from the delivery system by applying thermal or electrolytic power or by activating a mechanical detachment mechanism.

One widely used embolic device is a coil system including soft, helically wound coils. Three-dimensional microcoils had been developed for use especially in treating wide-necked aneurysms. Three-dimensional microcoils have a primary or linear configuration e.g., when stretched in a delivery system, and assume a secondary or three-dimensional configuration e.g., when deployed at the target site. Three-dimensional microcoils can provide adequate coverage across the wide neck of an aneurysm. Three-dimensional microcoils can also provide a frame within an aneurysm into which subsequent coils can be placed.

Three-dimensional microcoils are manufactured with shape-setting tools. One type of conventional shape-setting tools has a configuration which can produce a microcoil in a simple “corkscrew” shape. The end result is a microcoil that once deployed inside an aneurysm is expected to reconfigure itself to conform to the three-dimensional dome-shape of an aneurysm. However, there is no guarantee that the microcoil would reconfigure itself into a 3D shape as intended. Another type of conventional shape-setting tools has a configuration which can create a microcoil in a true 3D shape, but is traditionally limited in adding length to the overall coil via the number of sections in the shape-setting tool. That is, in order to create more length in the final microcoil product, more sections of the shape-setting tool are required.

Therefore, there remains a need for improved coil systems and methods of making the coil systems. It would be desirable to provide a shape-setting device or coil winding fixture that allows for making a 3D-shaped microcoil system with extended coil lengths for the same coil sizes. It would be desirable to provide a 3D coil system that has structural stability and allows for tighter packing of an aneurysm.

### SUMMARY

In one aspect, embodiments of the disclosure feature an embolic device. In general, an embodiment of the embolic device comprises a microcoil having a primary configuration. The microcoil comprises a first portion having a secondary configuration. The first portion in the secondary configuration comprises one or more sets of loops. Loops of each of the one or more sets intersect one with another in succession in a cycle, collectively forming a three-dimensional shape.

In various embodiments of the aspect, the loops of each of the one or more sets intersect at middle sections of the loops of each of the one or more sets along the cycle.

In various embodiments of the aspect, the one or more sets of loops comprise a first set of loops and a second set of loops overlaying the first set of loops, wherein the loops of the first set and the loops of the second set are generally concentric. The loops of the second set may have a diameter greater than a diameter of the loops of the first set.

In various embodiments of the aspect, the embolic device comprises two to fourteen sets of loops, wherein the loops of the two to fourteen sets are generally concentric. The two to fourteen sets of loops may extend a length of the microcoil ranging from 20 to 400 mm.

In various embodiments of the aspect, at least one of the one or more sets of loops comprises a first loop, a second loop, and one or more intermediate loops between the first loop and the second loop, wherein the first loop, the second loop, and the one or more intermediate loops intersect one with another in succession in the cycle, collectively forming a generally spherical or ellipsoidal shape. The first loop, the second loop, and the one or more intermediate loops may comprise at least a complete loop and at least a loop consisting of two partial loops. In an embodiment, the first loop, the second loop, and the one or more intermediate loops comprise two complete loops and one or more loops consisting of two partial loops.

In various embodiments of the aspect, the microcoil may further comprise a second portion distal to the first portion, wherein the second portion of the microcoil has a secondary configuration comprising a loop in a generally circular shape having a diameter smaller than the diameter of the complete loop or of the loop consisting of two partial loops of the first portion. In an embodiment, the loop of the second portion of the microcoil is adjacent to the first loop of the first portion of the microcoil, wherein the first loop is a complete loop. In an alternative embodiment, the loop of the second portion of the microcoil is adjacent to the first loop of the first portion of the microcoil, wherein the first loop is a loop consisting of two partial loops.

In another aspect, embodiments of the disclosure feature a shape-setting device. In general, an embodiment of the shape-setting device comprises a three-dimensional (3D) body having a curved surface, and a plurality of winding members extending from the curved surface of the 3D body and arranged in a plurality of groups in a plurality of sections of the curved surface. Each of the plurality of groups of winding members is configured to allow a microcoil to wrap around to form a loop. Adjacent groups of winding members share a winding member to allow loops formed by the adjacent groups to intersect one with another.

In various embodiments of the aspect, the 3D body is generally in a spherical or ellipsoidal shape. The winding members of each of the plurality of groups may be arranged at intervals and have peripheries configured to allow the microcoil to wrap around to form a generally circular or elliptical loop. The winding members of each of the plurality of groups may comprise a first set of winding members arranged opposite to each other, and a second set of winding members arranged opposite to each other, wherein extension of outer peripheries of the winding members of the first set forms a generally circular or elliptical shape. In an embodiment, one of the winding members of the second set is shared with an adjacent group of winding members. In another embodiment, the winding members of the second sets of the plurality of groups may be located at an imaginary cycle. In an embodiment, the plurality of winding members may be arranged in two to twenty-four groups allowing the microcoil to wrap around to form two to twenty-four loops around the 3D body in a cycle.

In various embodiments of the aspect, the plurality of winding members are arranged in four groups allowing the microcoil to form four loops around the 3D body in a cycle, wherein the first set of winding members of each of the four groups comprises an outer periphery in a shape of a circular segment. In an embodiment, the outer periphery of the circular segment of the first set of winding members has a radius ranging from 1.5 mm to 10 mm respectively.

In various embodiments of the aspect, at least one of the plurality of groups of winding members further comprises a cylindrical winding member extending from the curved surface of the 3D body and surrounded by the first and second sets of winding members of the at least one of the plurality of groups, wherein the cylindrical winding member comprises a periphery allowing the microcoil to wrap around to form a generally circular loop having a diameter smaller than a diameter of a loop formed by the winding members of the at least one of the plurality of groups.

In another aspect, embodiments of the disclosure feature a method of making an embolic device. In general, an embodiment of the method comprises obtaining a microcoil and a shape-setting device comprising a three-dimensional (3D) body having a curved surface and a plurality of winding members extending from the curved surface and arranged in a plurality of groups, winding the microcoil on the winding members of the plurality of groups in a first cycle around the 3D body to form a first set of loops intersecting one with another in succession, and heating the microcoil on the shape-setting device to obtain an embolic device comprising the first set of loops with a configuration of a three-dimensional shape.

In various embodiments of the aspect, in the first cycle of winding the microcoil is wound a single loop on each of the plurality of groups of the winding members. The single loop may be a complete loop or a loop consisting of two partial loops. In an embodiment, the first set of loops comprises two complete loops.

In various embodiments of the aspect, the method further comprises winding the microcoil on the winding members of the plurality of groups in a second cycle around the 3D body to form a second set of loops intersecting one with another in succession, wherein the second set of loops overlays the first set of the loops and is generally concentrical with the first set of loops, and heating the microcoil on the shape-setting device to obtain the embolic device comprising the first set and the second set of loops. In an embodiment, in the second cycle of winding, the microcoil is wound a single loop on each of the plurality of groups of the winding members. The single loop in the second cycle of winding may be a complete loop or a loop consisting of two partial loops.

In various embodiments of the aspect, each of the first set and the second set of loops comprises at least two complete loops.

In various embodiments of the aspect, the winding step may be repeated in one to fourteen cycles.

This Summary is provided to introduce selected aspects and embodiments of this disclosure in a simplified form and is not intended to identify key features or essential characteristics of the claimed subject matter, nor is it intended to be used as an aid in determining the scope of the claimed subject matter. The selected aspects and embodiments are presented merely to provide the reader with a brief summary of certain forms the invention might take and are not intended to limit the scope of the invention. Other aspects and embodiments of the disclosure are described in the section of Detailed Description.

These and various other aspects, embodiments, features, and advantages of the disclosure will become better understood upon reading of the following detailed description in conjunction with the accompanying drawings.

### BRIEF DESCRIPTION OF THE DRAWINGS

FIG. 1A depicts a coil system according to embodiments of the disclosure.

FIG. 1B depicts the coil system of FIG. 1A in a different perspective view according to embodiments of the disclosure.

FIG. 2A depicts a coil winding fixture according to embodiments of the disclosure.

FIG. 2B depicts the coil winding fixture of FIG. 2A in a different perspective view according to embodiments of the disclosure.

FIG. 2C depicts the coil winding fixture of FIG. 2A in a further different perspective view according to embodiments of the disclosure.

FIG. 3 is an illustration of the coil winding fixture of FIG. 2A in a flattened, two-dimensional view.

FIGS. 4A-4G illustrate an example winding operation according to embodiments of the disclosure.

FIG. 5 illustrates an example winding operation according to embodiments of the disclosure.

FIG. 6 illustrates an example winding operation according to alternative embodiments of the disclosure.

FIG. 7 depicts an example coil winding fixture according to alternative embodiments of the disclosure.

### DETAILED DESCRIPTION OF EMBODIMENTS

### Overview

Embodiments of the disclosure provide an embolic device or a coil

system. The coil system can be made with a shape-setting device or coil winding fixture and has a true three-dimensional (3D) shape with varying lengths. The coil system has a unique winding pattern with concentrically deployed, intersecting loops that can be repeated multiple times to obtain the desired length. The winding pattern of the coil system can be designed to not overlap onto itself until a complete cycle is wound around the winding tool. The winding cycle can be repeated for additional lengths of the same primary microcoil diameter. By way of example, for a given coil size, e.g., 10 mm, varying lengths of a coil system can be made, e.g., 10 mm×30 mm, 10 mm×40 mm, 10 mm×50 mm, and so on. Increased lengths for the same coil size allows for more aneurysm packing with a single coil system delivery. This reduces the number of coils needed to achieve the target packing density for an aneurysm fill, which reduces the overall cost and procedure time.

The intersecting loops of the coil system provide structural stability and allow for tighter packing. Structural stability can be achieved by the outward force of the loop being deployed applied to an existing coil for part of the circumference. Tighter packing can be provided by minimized straight run between successive loops, because the more often that the coil changes direction, the more readily that it folds and packs. This is in contrast to conventional shape-setting tools which have a gap between each set of repeating features, resulting in a longer straight section of coil.

Being wrapped concentrically onto itself, the coil system of the disclosure naturally wants to form a 3D shape, e.g., a shape of a spherical or ellipsoidal body upon which it is wound. This is in contrast to conventional tools which necessitate adding additional length or features to the tool to attain longer lengths. The concentrical wrapping design has the added feature of increased radial force over the course of a single coil deployment. By way of example, successive wraps around the same winding tool may have a slightly larger diameter, e.g., two times of the diameter of the primary coil, by resting atop the previous coil. When eventually deployed within the aneurysm, the latter larger coil loops will be deployed within the smaller first loops, exerting more force than like-sized loops would. Increasing radial force over the length of a coil system will result in better apposition of the coil system to the inner wall of an aneurysm, effectively locking the coil in place or mitigating the concern for migration.

With reference to the figures, various embodiments of an embolic device, a shape-setting device, and a method of making an embolic device will now be described. It should be noted that the figures are intended for illustration of embodiments but not for exhaustive description or limitation on the scope of the disclosure. Alternative structures and components will be readily recognized as being viable without departing from the principle of the claimed invention.

### Coil System

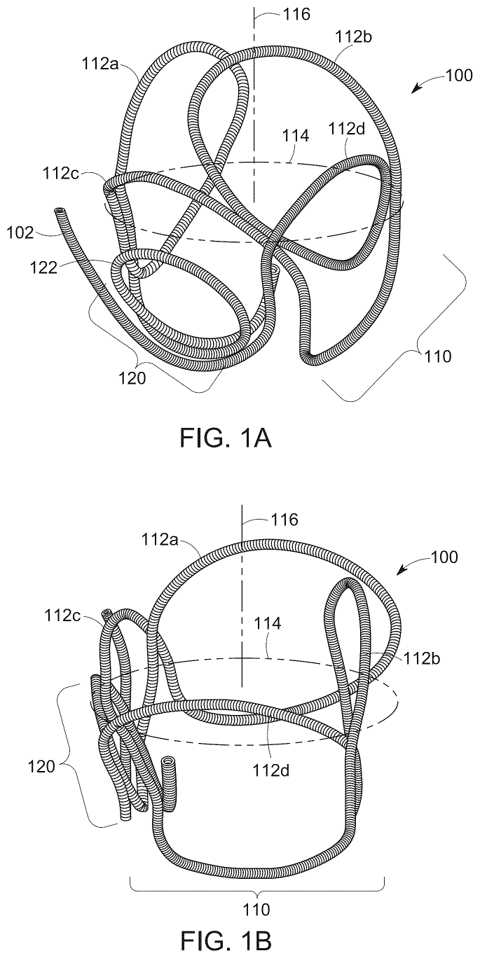

FIGS. 1A-1B depict an example coil system or embolic device 100 according to embodiments of the disclosure. In a broad overview, the example coil system 100 comprises a primary microcoil 102 having a first portion 110 in a three-dimensional shape. The first portion 110 may include one or more sets of loops 112a-d intersecting one with another in succession, collectively forming e.g., a generally spherical or ellipsoidal shape. For clarity of illustration, only one set of loops 112a-d of the first portion 110 is shown in FIGS. 1A-1B. The coil system 100 may also include a second portion 120 distal to the first portion 110. The second portion 120 may include an atraumatic loop 122. The loop 122 of the second portion 120 may have a dimension smaller than that of the loops 112a-d of the first portion 110. In use of the coil system 100, the first portion 110 and the second portion 120 may be disposed in a microcatheter in a linear or primary configuration for delivery of the coil system 100 to a target site. The second portion 120 can be disposed distal to the first portion 110 to allow the atraumatic loop 122 to be released first to minimize tissue damage during insertion and deployment of the coil system 100. When deployed at the target site, the first portion 110 of the coil system 100 assumes its secondary configuration in a three-dimensional shape as shown in FIG. 1A-1B. By way of example, in treating aneurysms the coil system 100 may assume a three-dimensional shape in apposition with an inner wall of an aneurysm.

With reference to FIGS. 1A-1B, the loops 112a-d of a set intersect one with another in succession in a cycle, as indicated at 114. For illustration purpose, FIGS. 1A-1B show a set including a first loop 112a, a second loop 112b, and intermediate loops 112c, 112d between the first loop 112a and the second loop 112d. As shown, the first loop 112a intersects with an intermediate loop 112c, which in turn intersects with another intermediate loop 112d, which in turn intersects with the second loop 112b. The second loop 112b then intersects with the first loop 112a, completing a cycle indicated at 114. The individual loops, such as loops 112a-d, may intersect or adjoin one with another in succession in a manner of surrounding an axis 116 collectively forming a three-dimensional shape. The individual loops 112a-d, which may be in a circular, elliptical, or other regular or irregular shape, can be either generally planar or curved. The intersecting of the generally circular or elliptical loops may occur at the middle sections of the loops as shown. According to embodiments of the disclosure, in a single complete cycle, the loops of a set intersect one with another, but do not overlap each other. As used herein, a loop “overlaps” with another loop if the majority portion or area of the loop, e.g., more than 50 percent, lays over the other loop. A loop “intersects” with another loop if the loop crosses the other loop but less than 50 percent of the area of the loop lays over the other loop. Loops intersecting one with another but not overlapping each other helps ensure the coil system to be properly distributed about the interior, e.g., of an aneurysm so that the deployed coil system does not favor a particular zone of the aneurysm.

FIGS. 1A-1B show four individual loops 112a-d in a set or cycle for clarity of illustration. It should be noted that a set of loops may include fewer or more than four loops. According to embodiments of the disclosure, two to twenty-four (2-24) individual loops may be present in a set or cycle, depending on medical applications.

FIGS. 1A-1B show only one set of loops 112a-d in the first portion 110 of the coil system 100 for clarity of illustration. According to embodiments of the disclosure, the first portion 110 of the coil system 100 may include two or more sets of loops. Each of the two or more sets may include a plurality of loops intersecting one with another in succession in a cycle, as discussed above. In the two or more sets, loops of one set may lay over loops of another set, forming “layers” or “wraps” of the first portion of the coil system. According to embodiments of the disclosure, the loops of the two or more sets are concentrical, e.g., about an axis 116. Depending on medical applications, the first portion 110 of the coil system 100 may include two to fourteen (2-14) sets or layers of loops. According to embodiments of the disclosure, the loops of an outer set or layer may be slightly larger than the loops of an inner set or layer. For example, the diameter of generally circular loops of an outer set may be greater than the diameter of generally circular loops of an inner set by two times of the diameter of the cross-section of the primary coil. The larger loops can be deployed within the proceeding smaller loops, exerting more force than like-sized loops would. Increasing radial force over the length of the coil system can result in better apposition of the coil system to the inner wall of the aneurysm, effectively locking the coil in place and mitigating the concern for migration. According to alternative embodiments of the disclosure, the size of the loops of an outer set may be approximately the same as the size of the loops of an inner set.

The loops 112a-d of a set may be a complete loop or full loop. The loops 112a-d of a set may also be a loop consisting of two partial or half loops. As will be discussed further below, a complete or full loop consists of a continuous section of the microcoil as contrast to a loop of two partial or half loops. In FIGS. 1A-1B, loops 112a-b are a complete or full loop whereas loop 112c-d are a loop consisting of two partial loops.

The primary microcoil 102 forming the coil system 100 of the disclosures can be made of a helically wound wire. The wire can be made of a metal, a metal alloy, or any other material suitable for forming an embolic device, including but not limited to platinum, platinum-tungsten alloy, platinum-iridium alloy. The wire may have a diameter ranging approximately from 0.001″ to 0.005″, and preferably from 0.001″ to 0.003″ for neurovascular applications. The primary microcoil 102 made of a helically wound wire may have a cross-sectional diameter ranging approximately from 0.008″ to 0.04″, and preferably from 0.008″ to 0.024″ for neurovascular applications. The coil system 100 or the first portion 110 of the coil system 100 formed with a primary microcoil 102 may have a size defined by the dimension of a loop. According to embodiments of the disclosure, the coil system 100 may have a size or diameter ranging from 3 mm to 20 mm, or from 3 mm to 15 mm for neurovascular applications.

### Coil Winding Fixture

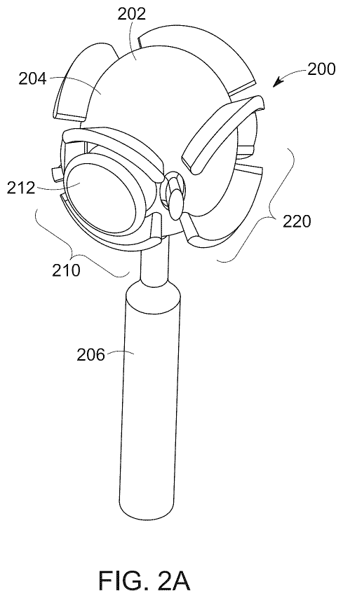

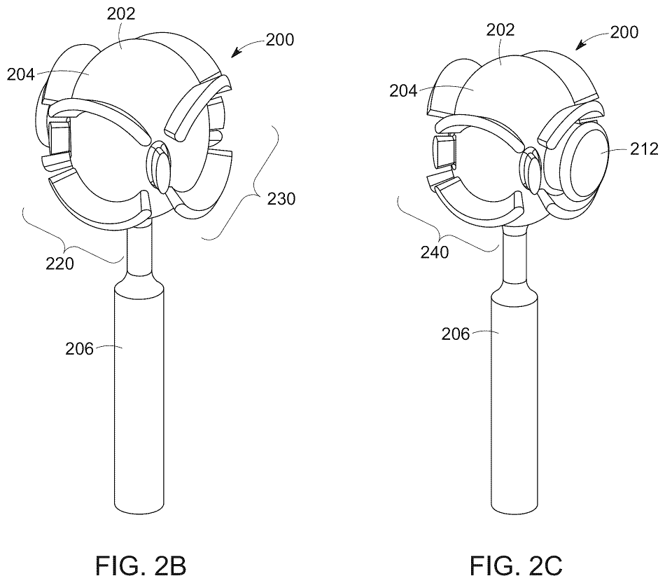

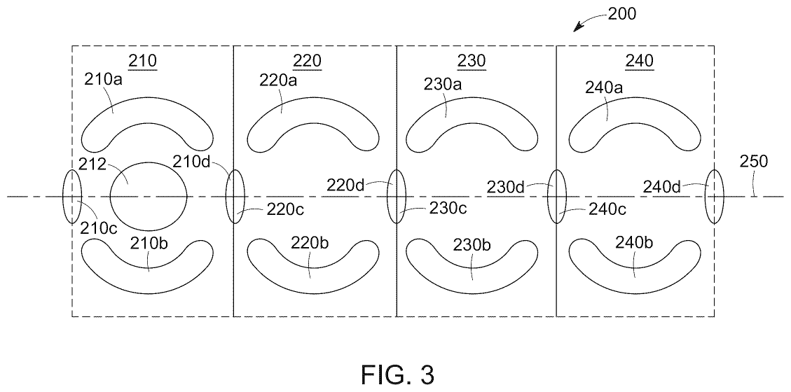

With reference to FIGS. 2A-2C and 3, a coil winding fixture or shape-setting device 200 according to one aspect of the disclosure is now described. FIGS. 2A-2C are perspective views of an example coil winding fixture 200. FIG. 3 is a flattened, two-dimensional illustration of the coil winding fixture 200 of FIGS. 2A-2C. As shown, the coil winding fixture 200 comprises a three-dimensional (3D) body 202 having a curved surface 204, and a plurality of winding members 210a-d, 220a-d, 230a-d, 240a-d (FIG. 3) extending from the curved surface 204 of the 3D body 202. The winding members 210a-d, 220a-d, 230a-d, 240a-d are arranged in a plurality of groups 210, 220, 230, 240 in a plurality of sections of the curved surface 204 of the 3D body 202. The winding members 210a-d, 220a-d, 230a-d, 240a-d of each of the plurality of groups 210, 220, 230, 240 are configured and/or arranged to allow a microcoil to wrap around to form a loop. The winding members of adjacent groups may share a winding member, e.g., winding member 210d/220c of adjacent groups 210 and 220, winding member 220d/230c of adjacent groups 220 and 230, winding member 230d/240c of adjacent groups 230 and 240, winding member 240d/210c of adjacent groups 240 and 210, to allow loops formed on the adjacent groups of winding members to intersect or adjoin one with another, as will be discussed further below.

The 3D body 202 provides a support for coil winding, and in conjunction with the winding members 210a-d, 220a-d, 230a-d, 240a-d, may provide a specific shape to the coil formed on the 3D body 202. The 3D body 202 may be generally in a spherical or ellipsoidal shape, or in any other regular or irregular shape having a curved surface. The 3D body 202 may be constructed of a material that can withstand a high heat setting temperature e.g., ranging from 400° C. to 1000° C. Example materials suitable for the 3D body 202 include high heat-resistant alloy materials such as tungsten-carbide, stainless steel, and other metal or metal alloys. The winding members 210a-d, 220a-d, 230a-d, 240a-d can be constructed of a heat resistant material, which can be the same as or different from the material of the 3D body 202. The winding members 210a-d, 220a-d, 230a-d, 240a-d can be provided on the 3D body 202 by any suitable methods. Alternatively, the winding members 210a-d, 220a-d, 230a-d, 240a-d and the 3D body 202 can be constructed of a single unitary body.

Winding members 210a-d, 220a-d, 230a-d, 240a-d can be configured and/or arranged to allow a microcoil to wrap around. Winding members 210a-d, 220a-d, 230a-d, 240a-d can be arranged in a plurality of groups around the curved surface 204 of the 3D body 202. For illustration purpose, the winding members 210a-d, 220a-d, 230a-d, 240a-d shown in FIGS. 2A-2C and 3 are arranged in four groups 210, 220, 230, 240 in four sections of the surface 204 of a spherical or ellipsoidal 3D body 202. It is apparent that fewer or more than four groups can be provided. In general, the winding members can be arranged in 2 to 24 groups depending on specific applications. FIG. 7 depicts an alternative embodiment of a coil winding fixture 700 comprising winding members arranged in three groups around the curved surface of a spherical or ellipsoidal body.

With reference to FIGS. 2A-2C and 3, the winding members 210a-d, or 220a-d, or 230a-d, or 240a-d of a group can be configured and/or arranged to allow a microcoil to wrap around to form a regular or irregular shape. The configuration and/or arrangement of the winding members 210a-d, or 220a-d, or 230a-d, or 240a-d of each of the groups may be the same, as depicted in FIGS. 2A-2C and 3. Alternatively, the configuration and/or arrangement of winding members of one group may be different from that of winding members of another group. For example, one group of winding members may be configured and/or arranged to allow a microcoil to wrap around to form a generally circular or elliptical loop, and another group of winding members may be configured and/or arranged to allow a microcoil to wrap around to form a non-circular loop, or a loop of other regular or irregular shape. In some embodiments, one group of winding members may be configured and/or arranged to allow forming a loop having a dimension same as or different from that of a loop formed by another group of winding members.

With reference to FIGS. 2A-2C and 3, according to embodiments of the disclosure, the winding members of a group may be arranged in sets at interval. By way of example, group 210 may include a first set of winding members 210a, 210b arranged opposite to each other and a second set of winding members 210c, 210d arranged opposite to each other. For illustration, FIGS. 2A-2C and 3 depict a first set or pair of winding members 210a, 210b in the form a circular segment arranged opposite to each other. The circular segments 210a, 210b may include an outer periphery having a radius. Extension of the outer periphery of the circular segments 210a, 210b may form a circle having the radius. A second set or pair of winding members 210c, 210d may be arranged opposite to each other. The second set or pair of winding members 210c, 210d may serve as “islands” separating the first set or pair of winding members 210a, 210b. Group 220, 230, and 240 winding members may have a configuration and/or arrangement same as or similar to that of group 210. The configuration and/or arrangement of winding members in pairs or sets allow adjacent groups of winding members to share a common winding member, thereby allowing loops formed by the adjacent groups to intersect one with another, to be discussed further below. The configuration and/or arrangement of winding members in pairs or sets also allow winding of the microcoil to form either a complete/full loop or a partial/half loop, as will be discussed further below.

With reference to FIGS. 2A-2C and 3, according to embodiments of the disclosure, each group of winding members 210, 220, 230, 240 may include a first set of winding members 210a-b, 220a-b, 230a-b, 240a-b and a second set of winding members 210c-d, 220c-d, 230c-d, 240c-d, wherein the second set of winding members 210c-d, 220c-d, 230c-d, 240c-d of all groups 210, 220, 230, 240 are located such that, if connected, would form a circle or ring, as indicated at 250 in FIG. 3. The first set of winding members 210a-b, 220a-b, 230a-b, 240a-b and the second set of winding members 210c-d, 220c-d, 230c-d, 240c-d may be arranged such that the circle or ring 250 formed by connecting the second set of winding members 210c-d, 220c-d, 230c-d, 240c-d would traverse the middle sections of a plurality of circles that would be formed by connecting respective groups of winding members 210, 220, 230, 240. As such, the loops formed by the microcoil on the winding fixture can intersect one with another in succession at the middle sections of the loops, to be discussed further below.

With reference to FIGS. 2A-2C and 3, according to embodiments of the disclosure, the coil winding fixture 200 may further include a cylindrical winding member 212 extending from the curved surface 204 of the 3D body 202. For example, the cylindrical winding member 212 may be located within or surrounded by a group e.g., group 210 of the winding members 210a-d. The cylindrical winding member 212 may have a periphery allowing the microcoil to wrap around to form a generally circular loop having a diameter smaller than the diameter of a loop formed by e.g., group 210 of winding members 210a-d. The cylindrical winding member 212 allows the microcoil to wrap around to form an atraumatic distal loop.

With reference to FIGS. 2A-2C, the coil winding fixture 200 may further include an elongate post 206 for securing the coil winding fixture 200. The elongate post 206 can be constructed of a material same as or different from the material of the 3D body 202 and/or the winding members 210a-d, 220a-d, 230a-d, 240a-d.

### Method Of Making Coil System

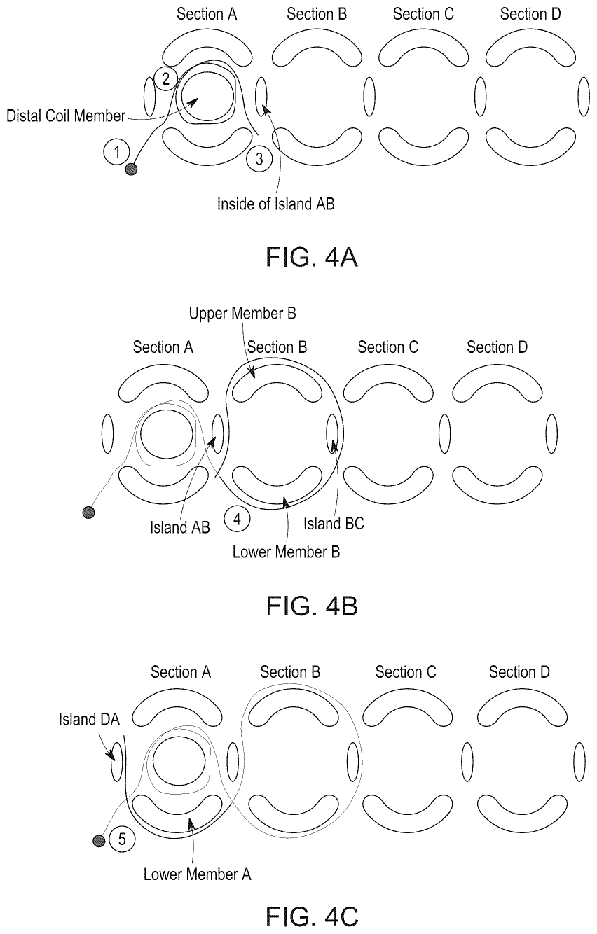

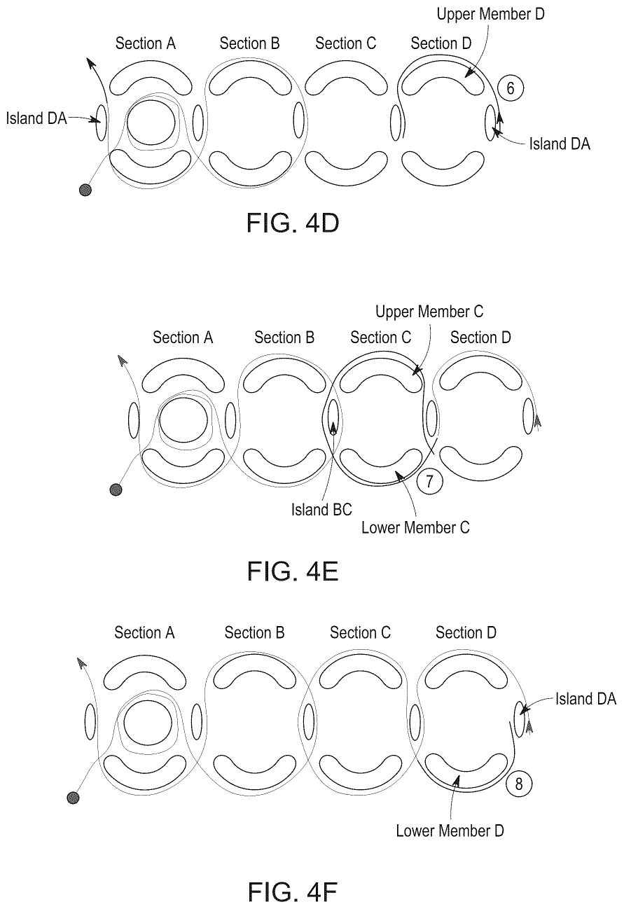

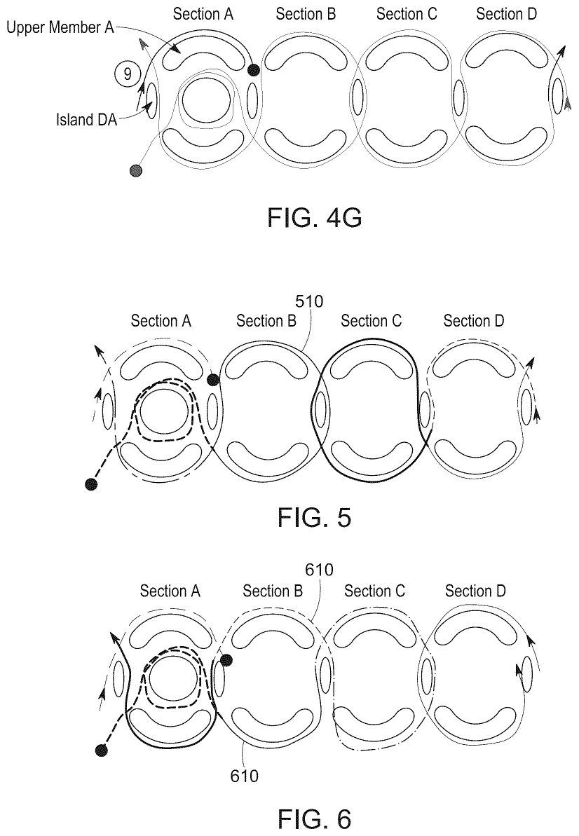

With reference to FIGS. 4A-4G, embodiments of a method of making a coil system or an embolic device according to one aspect of the disclosure is now described. In general, the method comprises obtaining a primary microcoil and a coil winding fixture or shape-setting device. The coil winding fixture may include a three-dimensional (3D) body having a curved surface and a plurality of groups of winding members extending from the curved surface and arranged in a plurality of groups. The method comprises winding the primary microcoil on the plurality of groups of winding members in a cycle around the 3D body to form a set of loops intersecting one with another in succession, and heating the microcoil wound on the fixture to obtain a coil system including a set of loops with a configuration of a three-dimensional shape.

The primary microcoil can be obtained from the market available from various manufacturers. The coil winding fixture can be one described above in conjunction with FIGS. 2A-2C and 3. Other coil winding fixtures can also be used. By way of example, the coil winding fixture may include a spherical or ellipsoidal body and a plurality of groups of winding members arranged in a plurality of sections of the curved surface of the spherical or ellipsoidal body.

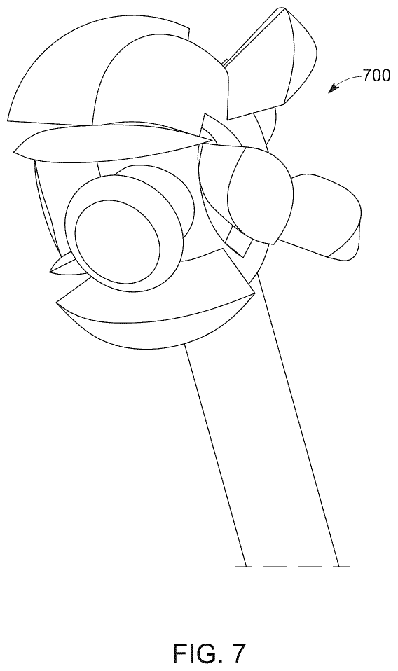

According to embodiments of the disclosure, the winding operation may include wrapping the microcoil on the plurality of groups of winding members in a cycle around the 3D body of the winding fixture to form a set of loops intersecting one with another in succession. According to embodiments of the disclosure, in the winding operation, a single loop of the microcoil is formed on each of the plurality of groups of the winding members in one cycle. In alternative embodiments, more than one loop e.g., one and half, two or more loops can be formed on each of the plurality of groups of the winding members in a cycle. As used herein, the term “single loop” refers to a loop formed by wrapping a microcoil on a group of winding members in one turn. A single loop can be a complete or full loop. A complete or full loop consists of a continuous portion of the microcoil. A single loop may also be a loop consisting of two partial loops, e.g., two half loops. In FIGS. 1A-1B, loops 112a and 112b are complete loops, whereas loops 112c and 112d are loops of two partial loops. Referring to FIG. 5, a complete or full loop 510 can be formed by wrapping a microcoil continuously on a single group of winding members (e.g., Section B) in one turn. Referring to FIG. 6, a loop of two partial loops 610 can be formed by wrapping a microcoil first on some of the winding members of a group (e.g., Section B), next wrapping the microcoil on winding members of another group (e.g., Section C), and then wrapping the microcoil on the remaining winding members of the group (e.g., Section B) to finish the loop 610.

According to embodiments of the disclosure, the winding operation may include wrapping the microcoil on the plurality of groups of winding members in two or more cycles around the winding fixture to form two or more sets of loops. By way of example, the winding operation may include winding the microcoil on the winding members of the plurality of groups in a first cycle around the 3D body to form a first set of loops intersecting one with another in succession, and winding the microcoil on the winding members of the plurality of groups in a second cycle around the 3D body to form a second set of loops intersecting one with another in succession. The second set of loops may lay over the first set of the loops and are generally concentrical with the first set of loops. The winding operation can be performed multiple cycles around the 3D body, to obtain a desired length of the embolic device.

With reference to FIGS. 4A-4G, an example winding operation according to embodiments of the disclosure is now described. The example winding operation is illustrated using a winding fixture including four groups of winding members arranged on the curved surface of an ellipsoidal body. FIGS. 4A-4G illustrate the winding fixture in a flattened, two-dimensional view. For clarity and simplifying description, terms “Section A,” “Section B,” “Section C,” and “Section D” are used to respectively refer to a group of winding members arranged in one of different sections of the curved surface of the ellipsoidal body. Terms “Upper Member” and “Lower Member” are used to refer to a first set of winding members disposed opposite to each other. Terms “Islands” are used to refer to a second set of winding members disposed opposite to each other and in between the Upper Member and Lower Member. Term “Island AB” refers to an island shared by Section A and Section B winding members. The term “Distal Coil Member” is used to refer to a winding member for forming a distal portion of the coil system. In FIGS. 4A-4G, numerals (1)-(9) represent sequential steps of the example winding operation.

With reference to FIG. 4A, the winding operation may start as follows:
(1). A primary microcoil is wrapped around the post of the winding fixture to fix it.
(2). Then the microcoil is wound around the Distal Coil Member to form an atraumatic distal loop.
(3). The microcoil is then wrapped on the inside of Island AB.

With reference to FIG. 4B, the winding operation may continue as follows:
(4). From the inside of Island AB, the microcoil is then wrapped around the Lower Member B, around the outside of Island BC and around Upper Member B to form a first full loop.

With reference to FIG. 4C, the winding operation may continue as follows:
(5). The microcoil is wrapped around the Lower Member A to the inside of Island AD to form a first half-loop.

With reference to FIG. 4D, the winding operation may continue as follows:
(6). The microcoil is wrapped from the outside of Island DA to around the Upper Member D, forming a second half-loop.

With reference to FIG. 4E, the winding operation may continue as follows:
(7). The microcoil is wrapped around the Lower Member C, up to the outside of Island CB, around the Upper Member C to inside of Island CD, forming a second full loop. The second full loop intersects the first full loop.

With reference to FIG. 4F, the winding operation may continue as follows:
(8). The microcoil is wrapped around the Lower Member D up to the inside of Island DA, forming a third half-loop.

With reference to FIG. 4G, the winding operation may continue as follows:
(9). The microcoil comes from the outside of Island AD and around Upper Member A, forming a fourth half-loop, completing a cycle around the ellipsoidal body of the winding fixture.

The winding operation may continue by wrapping the microcoil around the ellipsoidal body of the winding fixture two or more cycles, to attain a desired length of the coil system.

It should be noted that the winding operation discussed above in conjunction with FIGS. 4A-4G is provided for illustration. Many variations of the winding sequence are possible and the present claims are not limited to the example provided. For instance, in the example discussed above in conjunction with FIGS. 4A-4G, the winding operation starts with an atraumatic distal loop and then immediately follows a full loop in completing a wrapping cycle, as also shown in FIG. 5. In alternative embodiments, the winding operation may start with an atraumatic distal loop and then immediately follows a partial loop in completing a wrapping cycle, as illustrated in FIG. 6.

Various embodiments of a coil system, a coil winding fixture, and a method of making a coil system have been described. Advantageously, the coil winding fixture allows for making a true 3D-shaped coil system. Extended coil lengths for same coil sizes can be obtained. “Straight run” length between loops is reduced because groups or layers of loops can be concentrically wound or overlaid. The coil winding fixture and method can significantly reduce the cost of manufacturing in part by minimizing the number of tool sections required by conventional methods. The loops intersecting one with another in succession in a cycle provide structural stability and allow for tighter packing. The concentrically layered windings increase the radial force to provide improved aneurysm wall apposition.

In use, the embolic device of the disclosure can be delivered and deployed at a target site for treatment of a disorder within the vasculature of a patient such as an aneurysm using any suitable delivery device. The delivery device may include a detachment mechanism such as a mechanical, thermal or electrolytic power detachment mechanism to release the embolic device once delivered to the target site. A microcatheter may be first introduced to the target site through an access e.g., in the femoral artery or groin area of the patient by using an introducer sheath or guiding catheter. The microcatheter may be guided to the target site through the use of a guidewire. The guidewire is visible via fluoroscopy, allowing the microcatheter to be reliably advanced over the guidewire to the target site.

Once the target site has been accessed with the microcatheter tip, the guidewire can be withdrawn, clearing the lumen of the microcatheter. A system including a delivery device and an embolic device of the disclosure in a delivery configuration, can be placed into the proximal open end of the microcatheter and advanced through the microcatheter. When the embolic device reaches the distal end of the microcatheter, it can be detached from the delivery device by applying thermal or electrolytic power or by activating a mechanical detachment mechanism. Upon release, the embolic device assumes a three-dimension shape in the target site. The delivery device can be then removed from the microcatheter, and additional embolic device, if necessary for proper treatment, may be delivered and deployed in the same manner. After deployment of the implantable device, the microcatheter can be withdrawn from the vasculature of the patient.

Various embodiments of an embolic device for deployment within the vasculature of a human body and a method of making and/or using the embolic device are described are described with reference to figures. It should be noted that the figures are intended to facilitate illustration and some figures are not necessarily drawn to scale. Further, in the figures and description, specific details may be set forth in order to provide a thorough understanding of the disclosure. It will be apparent to one of ordinary skill in the art that some of these specific details may not be employed to practice embodiments of the disclosure. In other instances, well known components or process steps may not be shown or described in detail in order to avoid unnecessarily obscuring embodiments of the disclosure.

All technical and scientific terms used herein have the meaning as commonly understood by one of ordinary skill in the art unless specifically defined otherwise. As used in the description and appended claims, the singular forms of “a,” “an,” and “the” include plural references unless the context clearly dictates otherwise. The term “or” refers to a nonexclusive “or” unless the context clearly dictates otherwise. Relative terms such as above, below, top, bottom, up, down, under, over, upper, lower, horizontal, vertical, etc. are used for ease of illustration and discussion and not by way of limitation. The term “proximal” and its grammatically equivalent refers to a position, direction or orientation towards the operator or physician's side. The term “distal” and its grammatically equivalent refers to a position, direction or orientation away from the operator or physician's side. The term “first” or “second” etc. may be used to distinguish one element from another in describing various similar elements. It should be noted the terms “first” and “second” as used herein include references to two or more than two. Further, the use of the term “first” or “second” should not be construed as in any particular order unless the context clearly dictates otherwise.

Those skilled in the art will appreciate that various other modifications may be made. All these or other variations and modifications are contemplated by the inventors and within the scope of the invention.

1-12. (canceled)

13. A shape-setting device, comprising:
a three-dimensional (3D) body having a curved surface; and
a plurality of winding members extending from the curved surface of the 3D body and arranged in a plurality of groups in a plurality of sections of the curved surface, wherein:
each of the plurality of groups of winding members is configured to allow a microcoil to wrap around to form a loop; and
adjacent groups of winding members share a winding member to allow loops formed by the adjacent groups to intersect one with another.

14. The shape-setting device of claim 13, wherein the 3D body is generally in a spherical or ellipsoidal shape.

15. The shape-setting device of claim 14, wherein winding members of each of the plurality of groups are arranged at intervals and have peripheries configured to allow the microcoil to wrap around to form a generally circular or elliptical loop.

16. The shape-setting device of claim 15, wherein winding members of each of the plurality of groups comprise a first set of winding members arranged opposite to each other, and a second set of winding members arranged opposite to each other, wherein extension of outer peripheries of the winding members of the first set forms a generally circular or elliptical shape.

17. The shape-setting device of claim 16, wherein one of the winding members of the second set is shared with an adjacent group of winding members.

18. The shape-setting device of claim 16, wherein winding members of the second sets of the plurality of groups are located at an imaginary cycle.

19. The shape-setting device of claim 16 wherein the plurality of winding members are arranged in two to twenty-four (2-24) groups allowing the microcoil to wrap around to form two to twenty-four loops around the 3D body in a cycle.

20. The shape-setting device of claim 19, wherein the plurality of winding members are arranged in four groups allowing the microcoil to form four loops around the 3D body in a cycle, wherein the first set of winding members of each of the four groups comprises an outer periphery in a shape of a circular segment.

21. The shape-setting device of claim 20, wherein the outer periphery of the circular segment of the first set of winding members has a radius ranging from 1.5 mm to 10 mm respectively.

22. The shape-setting device of claim 16, wherein at least one of the plurality of groups of winding members further comprises a cylindrical winding member extending from the curved surface of the 3D body and surrounded by the first and second sets of winding members of the at least one of the plurality of groups, wherein the cylindrical winding member comprises a periphery allowing the microcoil to wrap around to form a generally circular loop having a diameter smaller than a diameter of a loop formed by the winding members of the at least one of the plurality of groups.

23-33. (canceled)
